(19) United States
(12) Patent Application Publication
(10) Pub. No.: US 2026/0354685 A1
(43) Pub. Date: Nov. 12, 2026

(54) MEDICAL INTAKE ASSISTANT DEVICE

(71) Applicant: Kimberley Kleckner, Apollo Beach, FL (US)

(72) Inventor: Kimberley Kleckner, Apollo Beach, FL (US)

(21) Appl. No.: 19/541,647

(22) Filed: Feb. 17, 2026

Related U.S. Application Data
(60) Provisional application No. 63/801,980, filed on May 8, 2025.

(51) Int. Cl. A61B 5/00; A61B 5/02

(52) U.S. Cl. CPC A61B 5/0002; A61B 5/02

(57) ABSTRACT

A medical intake assistant device is provided. The device automates the patient rooming process by performing routine intake tasks typically managed by medical assistants. It comprises a mobile platform with a motorized drive system, environmental sensors, and an autonomous navigation system for dynamic indoor movement. A patient identification module integrates facial recognition, RFID, barcode scanning, and short-range communication to verify identity. Upon identification, the device guides the patient to an exam room using a display, audio prompts, and directional lighting. It then collects vital signs via a weight scale, pulse oximeter, and blood pressure cuff, and prompts the patient to review medical history.

### CROSS-REFERENCE TO RELATED APPLICATION

The present application claims priority to, and the benefit of, U.S. Provisional

Application No. 63/801,980, which was filed on May 8, 2025, and is incorporated herein by reference in its entirety.

### FIELD OF THE INVENTION

The present invention relates generally to the field of medical services. More specifically, the present invention relates to a device designed to automate and optimize the patient rooming process by autonomously guiding patients to examination rooms, identifying patients through integrated recognition systems, and collecting vital signs and medical history data. Accordingly, the present disclosure makes specific reference thereto. Nonetheless, it is to be appreciated that aspects of the present invention are also equally applicable to other like applications, devices, and methods of manufacture.

### BACKGROUND

In outpatient clinical environments, medical staff are often burdened by a range of tasks that require both administrative precision and patient-facing interaction. Medical assistants, in particular, are tasked with duties such as escorting patients to examination rooms, measuring vital signs, reviewing medication histories, and preparing patients for provider evaluation. These responsibilities, while routine, are time-consuming and prone to error when performed under staffing constraints. The frequency of staff shortages caused by illness, vacation, or turnover contributes to workflow bottlenecks and delays in patient processing. Medical assistants are frequently interrupted in the middle of more complex clinical or administrative work to complete rooming procedures, which can lead to skipped steps, miscommunication, or inaccurate data entry. These interruptions may negatively affect patient safety, care consistency, and appointment efficiency. Additionally, patients may experience discomfort or privacy concerns during hurried or incomplete intake procedures. The accumulation of such inefficiencies can cause cascading delays throughout the clinical schedule, ultimately impacting the quality of care and staff morale. These challenges demonstrate the need for a reliable system that can support medical assistants, mitigate human error, and enhance the patient intake experience.

Therefore, there exists a long-felt need in the art for a medical intake assistant device that streamlines the patient rooming process to reduce workflow interruptions for clinical staff. There also exists a long-felt need in the art for a medical intake assistant device that ensures accurate and complete acquisition of patient data through standardized protocols. Moreover, there exists a long-felt need in the art for a medical intake assistant device that improves patient privacy and comfort during intake procedures in outpatient healthcare settings.

The subject matter disclosed and claimed herein, in one embodiment thereof, comprises a medical intake assistant device. The device is designed to automate and optimize the patient rooming process in clinical facilities by executing routine intake operations typically handled by medical assistants. The device may comprise a mobile platform supported by a motorized drive system mounted beneath a chassis. A control processor embedded within the chassis may receive real-time spatial data from an array of environmental sensors, and wherein an autonomous navigation system may govern real-time route planning and dynamic path adjustment, enabling the device to operate effectively in variable indoor environments. A patient identification module may be integrated into the device and configured to communicate with scheduling software, comprising facial recognition hardware, RFID readers, barcode scanners, and short-range communication systems. Upon positive patient identification, a guidance interface may be activated, consisting of a touch-capable display screen, multi-language audio output via embedded speakers, and directional lighting. The device may lead patients to an examination room and initiate a data collection sequence, utilizing an embedded weight scale, pulse oximeter, and automated blood pressure cuff to acquire vital signs. The display screen may prompt patients to verify or update historical data such as medical conditions and medication use. A data integration interface may transmit collected data to facility records.

In this manner, the medical intake assistant device of the present invention accomplishes all the foregoing objectives and provides a device that automates patient rooming tasks, thereby minimizing staff workload and reducing the risk of skipped procedural steps due to human oversight. Through integration of standardized protocols and reliable biometric and sensor-based measurements, the device further ensures accurate and consistent collection of vital health data and patient history. By facilitating private, patient-guided intake experiences and enabling seamless data transfer to clinical systems, the device addresses concerns related to workflow efficiency, staffing shortages, and patient comfort. As a result, the device offers a comprehensive solution to longstanding operational inefficiencies in outpatient care environments.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a medical intake assistant device. The device is designed to automate the patient rooming process in outpatient clinical environments by performing preparatory tasks typically conducted by medical assistants. The device autonomously escorts patients to examination rooms, collects vital signs, reviews medical history and medications, and notifies healthcare providers upon completion. The system is intended to improve workflow efficiency, reduce staff burden, minimize errors, and enhance patient privacy and experience.

The device is comprised of a mobile platform with a chassis mounted on a motorized drive system. Navigation of the device is managed by a control processor embedded within the chassis and supported by multiple environmental sensors. An autonomous navigation system governs route planning, localization, and real-time environmental mapping.

A patient identification module enables integration with facility scheduling systems. The module may be comprised of a facial recognition camera, RFID tag reader, barcode scanner, and short-range communication transceiver. A verification processor cross-references identifiers with appointment records for patient authentication.

Upon authentication, a guidance interface leads the patient to an examination room. The guidance interface is comprised of a display screen, speakers, and illuminated light strips. These components deliver directional, visual, and auditory cues, enabling accessibility for patients with various sensory or mobility needs.

Upon reaching the examination room, a vital sign acquisition sequence is initiated. This sequence includes an integrated weight scale for weight measurement, a pulse oximeter for oxygen saturation and pulse rate, and a blood pressure cuff for pressure readings. The display screen prompts the patient to review medical history and medication information.

Collected data is then transmitted through a data integration interface configured for real-time, encrypted synchronization with facility systems, ensuring data privacy and integrity. Upon completion of data collection, a notification is generated and sent to healthcare staff through the facility's communication systems.

Accordingly, the medical intake assistant device of the present invention is particularly advantageous as it automates patient rooming tasks, thereby minimizing staff workload and reducing the risk of skipped procedural steps due to human oversight. Through integration of standardized protocols and reliable biometric and sensor-based measurements, the device further ensures accurate and consistent collection of vital health data and patient history. By facilitating private, patient-guided intake experiences and enabling seamless data transfer to clinical systems, the device addresses concerns related to workflow efficiency, staffing shortages, and patient comfort. As a result, the device offers a comprehensive solution to overcome the longstanding operational inefficiencies and limitations in outpatient care environments.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a medical intake assistant device of the present invention in accordance with the disclosed architecture; and

FIG. 2 illustrates an enhanced perspective view of one potential embodiment of a medical intake assistant device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a medical intake assistant device that streamlines the patient rooming process to reduce workflow interruptions for clinical staff. There also exists a long-felt need in the art for a medical intake assistant device that ensures accurate and complete acquisition of patient data through standardized protocols. Moreover, there exists a long-felt need in the art for a medical intake assistant device that improves patient privacy and comfort during intake procedures in outpatient healthcare settings.

The present invention, in one exemplary embodiment, is comprised of a medical intake assistant device. The device is configured to execute preparatory tasks of the patient rooming process that are traditionally managed by medical assistants. By autonomously escorting patients to examination rooms, collecting vital signs, reviewing medical histories and medications, and alerting healthcare providers upon task completion, the device enhances workflow efficiency, reduces staff workload, minimizes human error, and improves both privacy and overall patient experience.

The device is comprised of a mobile platform, which includes a chassis mounted on a motorized drive system. A control processor embedded within the chassis manages navigation, utilizing data from various environmental sensors. An autonomous navigation system oversees route planning, localization, and real-time mapping of the clinical environment.

Integration of the device with facility scheduling systems is enabled by a patient identification module. The module is comprised of a facial recognition camera, RFID tag reader, barcode scanner, and short-range communication transceiver. Patient identity is authenticated through a verification processor that cross-references acquired identifiers with appointment records.

Following successful authentication, the patient is guided to an examination room via a guidance interface. The interface is comprised of a display screen, speakers, and illuminated light strips that provide visual, auditory, and directional cues, supporting accessibility for patients with diverse mobility or sensory requirements.

Upon arrival in the examination room, the device initiates a vital sign acquisition sequence. This sequence involves the use of an integrated weight scale for weight measurement, a pulse oximeter for determining oxygen saturation and pulse rate, and a blood pressure cuff for acquiring blood pressure data. The display screen is used to present medical history and current medication information for patient review. Data collected during this process is transmitted via a data integration interface that enables real-time, encrypted synchronization with facility digital systems, ensuring secure handling of patient information. Once all required information has been acquired, a notification is automatically generated and sent to clinical staff using the facility's existing communication infrastructure.

As such, the device provides a distinct advantage by automating the patient rooming workflow, thereby reducing staff burden and mitigating procedural errors arising from human oversight. Through standardized protocols and accurate sensor-based measurements, the device ensures reliable health data acquisition. Furthermore, by enabling private, patient-guided intake procedures and seamless data transfer, the device effectively addresses key challenges in outpatient settings, including inefficiency, staffing shortages, and patient discomfort, offering a comprehensive and autonomous operational solution.

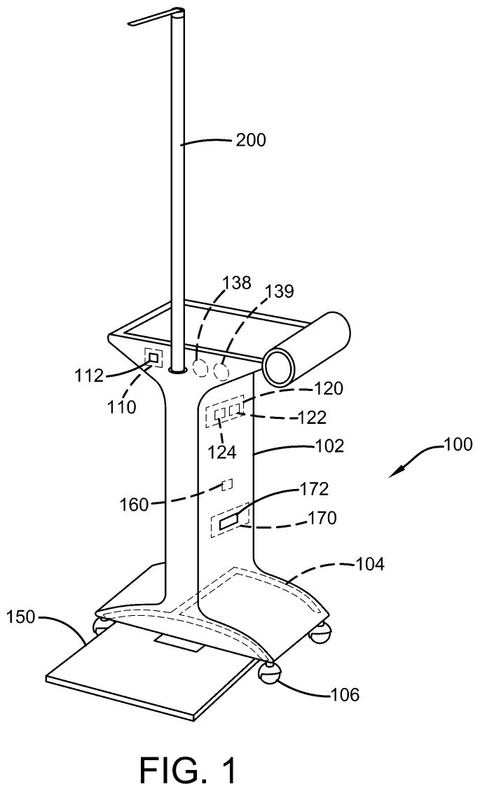

Referring initially to the drawings, FIG. 1 illustrates a perspective view of one potential embodiment of a medical intake assistant device 100 of the present invention in accordance with the disclosed architecture. The device 100 is designed to automate the patient rooming process in outpatient clinical environments by performing routine preparatory tasks typically handled by medical assistants. The primary purpose of the device 100 is to autonomously escort patients from the waiting area to an examination room, conduct preliminary data collection such as vital sign measurements, review the patient's medical history and medications, and notify healthcare providers when the patient is ready for evaluation. The device 100 is configured to improve workflow efficiency, reduce administrative burden on clinical staff, mitigate human error, and enhance patient privacy and experience.

The device 100 may be comprised of a mobile platform capable of autonomously navigating within clinical facilities. The device 100 may include a chassis 102 mounted on a motorized drive system 104. The motorized drive system 104 may be in the form of but is not limited to any combination of differential drive motors, omnidirectional wheel assemblies, or any combination of servo and stepper motor configurations. The wheels 106 may be comprised of any wheel type such as but not limited to rubberized, polyurethane-coated, or non-marking wheels to ensure stability and quiet operation across a range of floor surfaces including tile, linoleum, and carpet.

Steering and navigation of the device 100 may be governed by a control processor 110 that may be embedded within the chassis 102, as seen in FIG. 1. The control processor 110 may receive continuous inputs from multiple spatial and environmental sensors 112. The sensors 112 may include but are not limited to any combination of infrared proximity detectors for object avoidance, ultrasonic transceivers for short-range distance measurement, capacitive or mechanical tactile bumpers for collision detection, and optical floor sensors capable of detecting floor markers or contrasting patterns for navigational reference.

The device 100 may also be comprised of an autonomous navigation system 120 (as seen in FIG. 1) that may manage route planning, localization, and real-time environmental mapping. The navigation system 120 may be comprised of a processor 122 that may be supported by secondary hardware 124, which may include but is not limited to any combination of LiDAR units capable of 360-degree scanning, RGB cameras for visual landmark recognition, depth cameras for three-dimensional scene analysis, inertial measurement units (IMUs) for motion tracking, and gyroscopes for orientation control. The navigation system 120 may be operable in both static and dynamic environments with moving individuals or carts and may recalculate routes in response to changing corridor conditions or temporary obstructions.

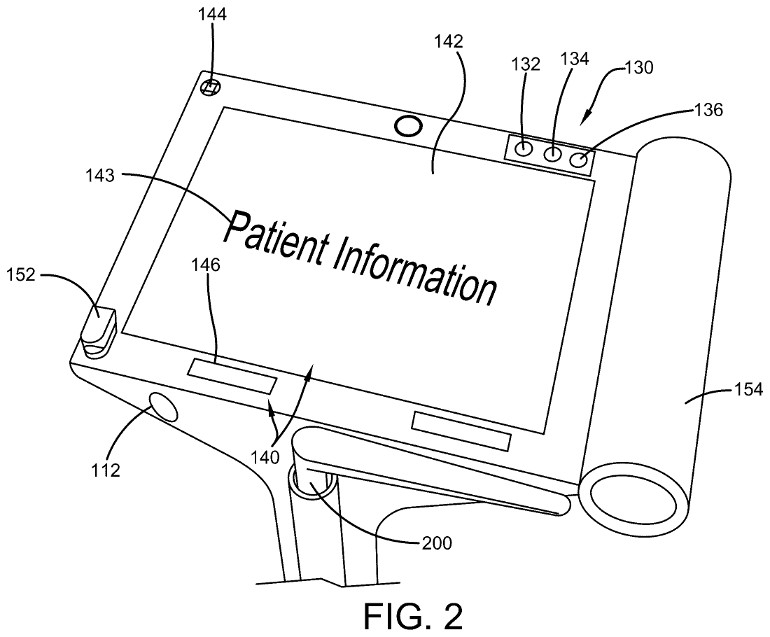

To identify and interact with individual patients, the device 100 may include a patient identification module 130 (as seen in FIG. 2) configured to interface with scheduling and check-in software of the facility in which the device is used in. The module 130 may be comprised of a facial recognition camera 132 capable of real-time face detection and matching. RFID tag readers 134 may be capable of reading active or passive tags embedded in patient ID cards or wristbands. Barcode scanners 136 may read one-dimensional or two-dimensional printed codes from appointment slips, identification badges, or mobile device screens. A short-range communication transceiver 138 may be included for proximity-based pairing of the device 100 with patient mobile devices using standards such as but not limited to Bluetooth, Bluetooth Low Energy (BLE), or Near Field Communication (NFC). A verification processor 139 may operate in conjunction with the identification module 130 to cross-reference acquired identifiers with facility appointment records and initiate patient engagement only upon a positive match.

Once a patient is identified and authenticated, the device 100 may activate a guidance interface 140 configured to lead the patient to a designated examination room (as seen in FIG. 2). The guidance interface 140 may be comprised of a display screen 142 capable of presenting (but not limited to) any combination of directional arrows, room numbers, or personalized greetings. The display screen 142 may be touch-sensitive and support capacitive, resistive, or optical input modalities to allow for user interaction when necessary. The guidance interface 140 may further include speakers 144 capable of delivering audio cues or voice instructions in multiple languages. Light strips 146 may be positioned along the perimeter of the chassis 102 or integrated into the display screen 142 and may illuminate in various colors or sequences to indicate movement direction, status changes, or to draw patient attention. The combination of visual, auditory, and tactile guidance may ensure accessibility for patients with varying levels of mobility, hearing, or vision.

Upon arrival in the examination room, the device 100 may initiate a vital sign acquisition sequence. The device 100 may incorporate an integrated weight scale 150 of any type that is used to acquire weight measurements discreetly as the patient stands on the scale 150 (shown in an extended configuration in FIG. 1). In a further embodiment, the device 100 is comprised of an adjustable measuring stick 200 used to measure the height of the patient while or while not standing on the device 100, as seen in FIG. 1. A pulse oximeter 152 of any type may be configured to acquire oxygen saturation and pulse rate. A blood pressure cuff 154 of any be present for measuring systolic and diastolic pressure calculation. The display screen 142 may prompt the patient to review displayed information 143 such as but not limited to information related to medical history and/or medication history.

Collected data from the device 100 may be transmitted through a data integration interface 160 configured for real-time synchronization with the digital systems of the facility. The data integration interface 160 may include wired and wireless communication transmitters such as but not limited to Wi-Fi, Ethernet, Bluetooth, and Zigbee. The integration interface 160 may operate in a secure, encrypted mode compliant with applicable data privacy standards to ensure the confidentiality and integrity of patient information. Once the patient has completed the vital sign acquisition sequence, the device 100 generates a notification which is sent to the healthcare staff using existing systems of the facility in the form of but not limited to a message, an email, a text, a page, etc.

All systems within the device 100 may be powered by a power system 170, as seen in FIG. 1. The power system 170 may be comprised of rechargeable battery cells such as but not limited to lithium-ion, lithium-iron-phosphate, or nickel-metal hydride chemistries. The device 100 may be recharged via a charging port 172 that may support inductive, conductive, or magnetic coupling mechanisms.

During use, the device 100 autonomously manages the patient rooming process in outpatient clinical environments. First, the device 100 navigates to the patient waiting area using the motorized drive system 104, which is mounted on the chassis 102 and supported by wheels 106 configured for smooth and quiet movement across various floor types. Navigation is directed by the control processor 110 and informed by spatial and environmental sensors 112 that detect obstacles, measure distances, and interpret floor patterns for accurate localization. The autonomous navigation system 120, supported by processor 122 and secondary hardware 124 that enables dynamic mapping and real-time route adjustments in environments with moving people or obstructions. Upon reaching the waiting area, patient identification is initiated through the patient identification module 130, which includes a facial recognition camera 132, an RFID tag reader 134, a barcode scanner 136, and a short-range communication transceiver 138. The verification processor 139 cross-references acquired identifiers with appointment records to confirm the identity of the patient. Following successful authentication, interface 140 engages to lead the patient to an assigned examination room using visual cues on the display screen 142, spoken instructions through speakers 144, and illuminated signals from light strips 146. Upon arrival in the examination room, vital sign collection begins. First, the patient stands on the integrated weight scale 150 for weight measurement, wherein the scale 150 may fold out from or slide into/out of the chassis 102. Then, the pulse oximeter 152 acquires oxygen saturation and pulse rate. Next, the blood pressure cuff 154 may be used to measure systolic and diastolic pressure. The display screen 142 then presents displayed information 143 including medical history and current medication lists for patient review and confirmation. All collected data is transmitted securely through the data integration interface 160, using encrypted communication protocols for real-time synchronization with the digital systems of the medical facility. Once all information is acquired and reviewed, a notification is then sent to healthcare staff indicating that the patient is ready for evaluation.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “medical intake assistant device” and “device” are interchangeable and refer to the medical intake assistant device 100 of the present invention.

Notwithstanding the foregoing, the medical intake assistant device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the medical intake assistant device 100 as shown in the FIGS. are for illustrative purposes only, and that many other sizes and shapes of the medical intake assistant device 100 are well within the scope of the present disclosure. Although the dimensions of the medical intake assistant device 100 are important design parameters for user convenience, the medical intake assistant device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A medical intake assistant device comprising:
chassis mounted on a motorized drive system;
a control processor configured to receive an input from a sensor;
an autonomous navigation system comprising a processor;
a patient identification module;
a display screen;
a speaker;
a weight scale;
an adjustable measuring stick;
a pulse oximeter;
a blood pressure cuff; and
a power system comprising rechargeable battery cells and a charging port.

2. The medical intake assistant device of claim 1 further comprised of a wheel attached to the motorized drive system.

3. The medical intake assistant device of claim 1, wherein the sensor is comprised of an infrared proximity detector, an ultrasonic transceiver, a capacitive tactile bumper, a mechanical tactile bumper, or an optical floor sensor.

4. The medical intake assistant device of claim 1, wherein the secondary hardware is comprised of a LiDAR unit, an RBG camera, a depth camera, an inertial measurement unit, or a gyroscope.

5. The medical intake assistant device of claim 1, wherein the patient identification module is comprised of a facial recognition camera.

6. The medical intake assistant device of claim 1, wherein the patient identification module is comprised of an RFID tag reader.

7. The medical intake assistant device of claim 1, wherein the patient identification module is comprised of a barcode scanner.

8. A medical intake assistant device comprising:
a chassis mounted on a motorized drive system;
a control processor configured to receive an input from a sensor;
an autonomous navigation system comprising a processor and a secondary hardware;
a patient identification module;
a display screen;
a speaker;
a weight scale;
a pulse oximeter;
a blood pressure cuff; and
a power system comprising rechargeable battery cells and a charging port.

9. The medical intake assistant device of claim 8 further comprised of a verification processor.

10. The medical intake assistant device of claim 8, wherein the display screen is comprised of a touch screen.

11. The medical intake assistant device of claim 8, wherein the sensor is comprised of an infrared proximity detector, an ultrasonic transceiver, a capacitive tactile bumper, a mechanical tactile bumper, or an optical floor sensor.

12. The medical intake assistant device of claim 8, wherein the secondary hardware is comprised of a LiDAR unit, an RBG camera, a depth camera, an inertial measurement unit, or a gyroscope.

13. The medical intake assistant device of claim 8, wherein the patient identification module is comprised of a facial recognition camera.

14. A medical intake assistant device comprising:
a chassis mounted on a motorized drive system;
a control processor configured to receive an input from a sensor;
an autonomous navigation system comprising a processor and a secondary hardware;
a patient identification module;
a display screen;
a speaker;
a light strip;
a weight scale;
a pulse oximeter;
a blood pressure cuff; and
a power system comprising rechargeable battery cells and a charging port.

15. The medical intake assistant device of claim 14, wherein the light strip indicates a direction of movement of the medical intake assistant device.

16. The medical intake assistant device of claim 14, wherein the display screen displays an information comprised of a medical history or a medication history.

17. The medical intake assistant device of claim 14 further comprised of a data integration interface.

18. The medical intake assistant device of claim 14, wherein the sensor is comprised of an infrared proximity detector, an ultrasonic transceiver, a capacitive tactile bumper, a mechanical tactile bumper, or an optical floor sensor.

19. The medical intake assistant device of claim 14, wherein the secondary hardware is comprised of a LiDAR unit, an RBG camera, a depth camera, an inertial measurement unit, or a gyroscope.

20. The medical intake assistant device of claim 14, wherein the patient identification module is comprised of a facial recognition camera.
